(19) United States
(12) Patent Application Publication
(10) Pub. No.: US 2026/0097044 A1
(43) Pub. Date: Apr. 9, 2026

(54) ION PAIR LIQUID CRYSTAL FORMULATIONS FOR LONG-ACTING INJECTABLES OF POORLY SOLUBLE DRUGS

(71) Applicant: FORDOZ PHARMA Corp., East Windsor, NJ (US)

(72) Inventors: Yuan Chen, Monmouth Junction, NJ (US); Sydney Ugwu, North Brunswick, NJ (US); Yiwen Wang, Plainsboro, NJ (US); James He, Green Brook, NJ (US)

(73) Assignee: FORDOZ PHARMA Corp., East Windsor, NJ (US)

(21) Appl. No.: 19/338,302

(22) Filed: Sep. 24, 2025

Related U.S. Application Data
(60) Provisional application No. 63/704,625, filed on Oct. 8, 2024.

(51) Int. Cl. A61K 31/5415; A61K 31/198; A61K 31/445; A61K 31/505; A61K 31/519; A61K 47/10; A61K 47/14; A61K 47/20; A61K 47/22; A61K 47/24

(52) U.S. Cl. CPC A61K 31/5415; A61K 31/198; A61K 31/445; A61K 31/505; A61K 31/519; A61K 47/10; A61K 47/14; A61K 47/20; A61K 47/22; A61K 47/24

(57) ABSTRACT

This application discloses a method to solubilize poorly soluble drugs in a liquid crystal vehicle for long-acting injectables. Poorly soluble acidic drugs-hydrophobic basic compounds ion pairs are incorporated into liquid crystal vehicles to solubilize and slowly release the drug after parenteral administration. Ion pairs such as Meloxicam-Dodecylamine, Sulfadiazine-Dodecylamine, Methotrexate-Dodecylamine, Levothyroxine-Dodecylamine, Meloxicam-Tridodecylamine, and Meloxicam-Bupivacaine are incorporated into liquid crystal vehicles to solubilize and control release the drugs.

This application claims the benefit of U.S. Provisional Patent Application Ser. No. 63/704,625 filed on Oct. 8, 2024, the contents of which are incorporated herein by reference.

### FILED OF THE INVENTION

The present disclosure relates to sustained-release liquid crystal compositions of poorly soluble drugs. Examples of poorly water-soluble drugs that may be used in the sustained-release liquid crystal compositions described herein are any poorly soluble drug that is known to treat conditions or diseases such as pain, psoriasis, systemic lupus erythematosus, mycosis fungoides, dermatomyositis, Pityriasis rubra pilaris, eczema, and the treatment of infections. In certain embodiments, the sustained-release liquid crystal compositions contain one or more poorly soluble drugs that are suitable for treatment and prevention of post-operative pain following soft tissue surgery or orthopedic surgery, joint pain, and inflammation, such as osteoarthritis and rheumatoid arthritis in humans and other mammalians. In other embodiments, the sustained-release liquid crystal compositions contain one or more poorly soluble drugs that are used to treat psoriasis, systemic lupus erythematosus, mycosis fungoides, dermatomyositis, Pityriasis rubra pilaris, eczema, and the treatment of infections. In further embodiments, the sustained-release liquid crystal compositions contain one or more poorly soluble drugs that are used to treat thyroid conditions such as hypothyroidism. The present invention also relates to methods of preparing, methods for testing, and methods of using the sustained-release liquid crystal compositions.

### BACKGROUND

Long-acting or sustained-release injectables are novel delivery systems that allow for the gradual release of medication into the bloodstream. To achieve an extended therapeutic effect, long-acting injectables are often formulated in polymeric systems. A significant number of drugs are hydrophobic and poorly soluble in water, which makes it difficult to formulate them for injectable administration.

Lyophobic liquid crystals are an amphiphilic mixture that behaves both like a liquid and a solid crystal. As reported in the U.S. Pat. No. 8,545,832, the liquid crystal phase can be formed by mixing water or body fluid with a composition of lipids that can form reverse hexagonal or reverse cubic phases. The liquid crystal phase can form a drug depot upon administration for long-acting injectables because of the unique channeling phase structure. However, in the patent, the inventors failed to provide a method to dissolve the poorly soluble drugs, i.e. the drugs that cannot be well dissolved in aqueous or in organic solvents in high concentrations. Hence, these drugs can only be formulated as micro- or nano-suspensions for parenteral delivery. The drawback of suspension formulation is that the drug release is minimal when injected into the subcutaneous layer. In addition, drug suspensions are more difficult to manufacture than drug solutions because of the need to control particle size distribution during processing. Although these drugs can be dissolved in large volumes of solvent, crystallization may occur upon dilution in an aqueous medium and result in poor drug release and/or absorption.

Pain management medications are drugs used to relieve the unpleasant feeling caused by tissue damage. Non-steroidal anti-inflammatory drugs (NSAIDs) are a class of drugs that exhibit both analgesic and anti-inflammatory effects. Compared to opioid analgesics, NSAIDs are safer and less addictive. However, some of the NSAIDs are poorly soluble in water and have limited solubility in organic solvents such as ethanol (EtOH), chloroform, and acetone.

U.S. Pat. No. 11,331,323 discloses a method to solubilize meloxicam (cyclooxygenase-2 selective inhibitor) with β-cyclodextrin via the inclusion complexation of meloxicam into the cyclodextrin cavity. However, cyclodextrin has several cell membrane effects and will cause membrane structure and permeability changes, which can lead to cell death at higher concentrations.

Another way of overcoming the insolubility issue of poorly soluble drugs is to use cosolvents or other chemicals to help solubilize the drugs. For example, International Patent Application No. WO 2008/062274 teaches the use of N-methyl 2-pyrrolidone (NMP), ethanol, and water to dissolve meloxicam. However, the combined solvent system cannot solubilize meloxicam at clinically significant concentrations.

U.S. Pat. No. 9,956,288 discloses the use of meglumine to dissolve meloxicam in water. Meglumine is a hydrophilic base and therefore forms meloxicam-meglumine salt and increases water solubility to 10 mg/ml. Other NSAID salts such as diclofenac epolamine, and methenamine sodium salicylate have been used for topical and oral delivery, respectively.

The hydrophobic ion pair approach has been used in the formulation of water-insoluble drugs. However, the hydrophobic ion pair dissociates readily in an aqueous medium, leading to drug precipitation. Hence, hydrophobic drug ion pairs can only exist as a suspension in an aqueous solution. An example of a commercially available hydrophobic drug ion pair dosage form is REBALANCE®, an antiprotozoal oral suspension with a hydrophobic ion pair of pyrimethamine and sulfadiazine in aqueous solution.

Canadian Patent No. 2,888,711, discloses a method using anionic anchoring agents to enhance the sustained release of cationic pharmacologically active substances in a sorbitan unsaturated fatty acid liquid crystal preconcentrate. However, in this patent, the bioactive ingredients are limited to cationic drugs. Furthermore, the patent did not disclose the use of the ion-pair effect to adjust the solubility of the drug in a liquid crystal formulation.

Accordingly, there is a need to increase the solubility of the poorly soluble bioactive ingredients, especially anion (or acidic) drugs, in a liquid crystal formulation of long-acting or sustained release injectables to achieve a high bioactive ingredient concentration required for parenteral injection via subcutaneous or intramuscular route, which often requires a low volume dosage form for administration via these routes.

### SUMMARY OF THE INVENTION

The present invention relates to liquid crystal compositions, including stable liquid crystal formulation precursors, stable liquid crystal preformulations, and injectable liquid crystal formulations, for the in-situ controlled or sustained delivery of poorly soluble drugs from the liquid crystal medium. The present invention also relates to sustained release compositions and corresponding precursor formulations, preformulations, and final injectable formulations containing one or more highly concentrated poorly soluble drugs in a liquid crystal state.

One aspect of the present invention is directed to an ion pair interaction (or Lewis acid-base pair, hydrogen bonding) between an acidic bioactive agent, pharmaceutical active ingredient or drug and a basic bioactive agent, pharmaceutical active ingredient, drug or excipient(s), which will increase the solubility of the bioactive agent, pharmaceutical active ingredient or drugs in the liquid crystal compositions and release the bioactive agent, pharmaceutical active ingredient or drug in a sustained manner after administration to a patient or subject.

In some embodiments, the ion pair interaction can be formed between acidic drugs and basic drugs, and/or between acidic drugs and basic excipient(s). In certain aspects of this embodiment, the acidic drug(s) have a lower pKa than their base counterparts.

The acidic bioactive agent, pharmaceutical active ingredient, or drug can be a bioactive ingredient(s), pharmaceutical active ingredient(s), or drug(s) that shows the property of a Lewis acid. Non-limiting examples of the bioactive agent(s), pharmaceutical active ingredient(s), or drug(s) that can work as an acid in the ion pair interaction are the poorly soluble NSAIDs, such as methotrexate, sulfadiazine, levothyroxine, and phenytoin, an acid form of a local anesthetic, levothyroxine, and a mixture of the foregoing.

In some embodiments, the poorly soluble NSAID that may be used in the present invention is selected from the group of NSAIDs that are slightly soluble, very slightly soluble, practically insoluble, or insoluble in water as defined by the United States Pharmacopeia. This includes etodolac, diflunisal, mefenamic acid, cataflam, indomethacin, piroxicam, meloxicam, oxaprozin, fenoprofen, salsalate, celecoxib, nabumetone, sulindac, and ketoprofen, and combinations of the foregoing.

In some embodiments, the acid forms of a local anesthetic that may be used in the present invention are selected from the group of bupivacaine hydrochloride, ropivacaine hydrochloride, lidocaine hydrochloride, procaine hydrochloride, chloroprocaine hydrochloride, tetracaine hydrochloride, cocaine hydrochloride, benzocaine hydrochloride, and combinations of the foregoing.

The basic bioactive agent(s), pharmaceutical active ingredient(s), or drug(s) can be bioactive ingredients that show the property of a Lewis base. Non-limiting examples of the bioactive agent(s), pharmaceutical active ingredient(s) or drug(s) that can be used as the base part of the ion pair interaction are a free base form of a local anesthetic selected from the group consisting of bupivacaine, ropivacaine, lidocaine, procaine, chloroprocaine, tetracaine, cocaine, benzocaine, and combinations of the foregoing.

In some embodiments, an excipient may be used as the base part of the ion pair interaction. Non-limiting examples of such basic excipients that can be used as the base part of the ion pair interactions can be organic bases with a pKa higher than their acidic bioactive agent(s), pharmaceutical active ingredient(s), or drug(s) counterparts. Examples of a few preferred organic bases that may be used in the present invention include, but are not limited to, bio-tolerated amines with an aliphatic chain with at least 4 carbons, such as dodecylamine, tridodecylamine, didodecylamine, n-octylamine, di-n-octylamine, tri-n-octylamine, tri-n-hexylamine, di-n-hexylamine, and combinations of the foregoing. If the bioactive ingredient(s), pharmaceutical active ingredient(s), or drug(s) are hydrophobic, the basic excipients can be high pKa ions, including but not limited to a pamoate salt, a deoxycholate salt, or combinations thereof.

Another embodiment of the present invention is directed to an injectable formulation, an injectable precursor formulation or an injectable preformulation, which can be a liquid crystal formulation composition compromising: an ion pair comprising at least one bioactive agent, pharmaceutical active ingredient or drug, at least one phospholipid, one or more low hydrophilic-lipophilic balance (HLB) lipids other than the phospholipid, and one or more bio-tolerated organic solvents. In some embodiments, the injectable formulation, injectable precursor formulation, or injectable preformulation may also further comprise a release adjuster or release controlling agent, water, and other pharmaceutically acceptable excipients.

In one aspect of the above liquid crystal compositions, the ion pair is formed by acidic and basic bioactive ingredients, pharmaceutical active ingredients, or drugs. The said acidic bioactive ingredients, pharmaceutical active ingredients, or drugs can comprise about 0.1% to about 20% by weight of the composition, and said basic bioactive ingredients, pharmaceutical active ingredients, or drugs can comprise about 0.1% to about 20% by weight of the composition.

In a further aspect of the above liquid crystal compositions, the ion pair is formed by at least one acidic bioactive ingredient, pharmaceutical active ingredients, or drugs, and one or more basic excipient(s). The said acidic bioactive ingredients, pharmaceutical active ingredients, or drugs can comprise about 0.1% to about 20% by weight of the composition, and one or more basic excipients can comprise about 0.1% to about 20% by weight of the composition.

In some embodiments of the injectable formulation or precursor formulations, the wt. % of the acidic counter ion in the liquid crystal injectable formulation, such as NSAIDs, can reach up to 20 wt. %, and the basic counter ion in the liquid crystal injectable formulation, such as the free base of a local anesthetic, can reach up to 20 wt. %, which should meet the need for the small volume for the administration of long-acting release formulation. In a previous commercial product, ZYRELEF®, the amount of the acidic NSAIDs was only 0.085 wt. %, and the basic local anesthetic was 2.85 wt. %, which are dissolved by polymers and organic solvents.

In some embodiments of the liquid crystal compositions, the phospholipid content ranges from about 5 wt. % to about 99 wt. %, preferably about 7.5 wt. % to about 90 wt. %, more preferably about 10 wt. % to about 85 wt. % and most preferably about 12 wt. % to 80 wt. % based on the overall weight of the injectable formulation, precursor formulation or preformulation.

In some embodiments of the liquid crystal compositions, the low HLB lipid comprises from 0 wt. % to 80 wt. %, preferably about 2.5 wt. % to about 70 wt. %, more preferably about 5 wt. % to about 60 wt. % and most preferably about 7.5 wt. % to 50 wt. % based on the overall weight of the injectable formulation, precursor formulation, or preformulation. Herein, the low HLB lipid means a lipid with an HLB below 12 and is not a phospholipid.

In some embodiments, the liquid crystal compositions may comprise 0 wt. % to 99 wt. %, preferably about 2.5 wt. % to about 70 wt. %, more preferably about 5 wt. % to about 60 wt. % and most preferably about 7.5 wt. % to 50 wt. % of one or more bio-tolerant organic solvents based on the overall weight of the injectable formulation, precursor formulation, or preformulation. The bio-tolerant organic solvent should dissolve the phospholipid, low HLB lipid, and/or ion pairs. The bio-tolerant organic solvents are pharmaceutically acceptable and, more preferably, pharmaceutically acceptable injectable solvents. Non-limiting examples of the bio-tolerant organic solvent include but are not limited to methanol, ethanol, isopropanol, n-butanol, t-butanol, benzyl alcohol, benzyl benzoate, N-methyl-2-pyrrolidone (NMP), polyethylene glycol 400 (PEG400), propylene glycol (PG), dimethylsulfoxide (DMSO), dimethylacetamide (DMAC), or a combination of the foregoing.

In some embodiments, the liquid crystal compositions may further comprise 0 wt. % to 50 wt. %, of a release adjuster or control release agent based on the overall weight of the injectable formulation, precursor formulation, or preformulation.

Another aspect of the present invention is directed to methods for preparing the liquid crystal injectable formulations or precursor formulations. One method that may be used is a one-pot method comprising the steps of: (i) dissolving one or more phospholipids in one or more bio-tolerant organic solvents to form a clear solution; and (ii) combining the clear solution with one or more low HLB lipids, at least one Lewis acid-base pair wherein the Lewis acid base pair comprises at least one poorly soluble bioactive agent, pharmaceutical active ingredient or drug to form a liquid crystal composition. The method may further comprise a step of sterilizing the liquid crystal composition by any known method, such as by filtration, radiation, heat/steam, or a combination of the foregoing.

In one embodiment of the method, sterilization of the liquid crystal composition comprises filtering the liquid crystal composition through a filter membrane having a pore size of about 0.2 μm or less.

A further aspect of the present invention is directed to an in-vitro dissolution test method for the injectable formulation, precursor formulation or preformulation of the invention comprising the steps of: (i) preparing a dissolution media by mixing a type of organic compound with a buffered aqueous solution and (ii) using a formulation or depot-holding container with a defined shape to hold the injectable formulation, precursor formulation or preformulation comprising the liquid crystal.

In one of the embodiments of the in-vitro dissolution test method, the organic compound composition of the dissolution media is chosen from the group of methanol, ethanol, cyclodextrin, PEG400, and a mixture of more than two thereof, which takes up 0 wt. % to 10 wt. % of the dissolution media.

In some embodiments of the in-vitro dissolution test method, the depot-holding container may be chosen from the group consisting of hydroxypropyl methylcellulose (HPMC) capsules, immersion cells, and dialysis bags. Suitable apparatus that may be used for carrying out the in-vitro dissolution test method of the present invention include, but are not limited to, known dissolution apparatus, for example, those described in USP 24, such as Apparatus 1 (basket test), Apparatus 2 (paddle test), and modifications thereof.

In an aspect, the content of the water in the injectable formulation or precursor formulation comprising the liquid crystal composition ranges from 0 wt. % to 20 wt. %. In some aspects, the water content is about 1, 2, 3, 4, 5, 6, 7, 8, 9, 10, 11, 12, 13, 14, 15, 16, 17, 18, 19, 20 wt. % or any range within the aforementioned values. If water content is above 0 wt. % in the final injectable formulation, water may be provided in a sealed container separately from the formulation, i.e., the precursor formulation or preformulation. In this embodiment, the administration of the final injectable formulation is performed after mixing the provided water with the precursor formulation or preformulation comprising the liquid crystal composition.

In some of the embodiments, the liquid crystal compositions of the present invention are administered intravenously, intramuscularly, topically, or preferably subcutaneously.

### BRIEF DESCRIPTION OF THE DRAWINGS

FIG. 1 illustrates the ternary diagram for the solubilization of meloxicam in the preformulated liquid crystal solution with increasing amounts of dodecylamine (Liquid crystal solution composition is shown in Example 5).

FIG. 2 illustrates the ternary diagram for the solubilization of sulfadiazine in the preformulated liquid crystal solution with increasing amounts of dodecylamine (Liquid crystal solution composition is shown in Example 5).

FIG. 3 illustrates the ternary diagram for the solubilization of methotrexate in the preformulated liquid crystal solution with increasing amounts of dodecylamine (Liquid crystal solution composition is shown in Example 5).

FIG. 4 illustrates the in-vitro release profile of meloxicam from Formulations #5 and #7 into Phosphate Buffered Solution (PBS), pH=7.4 (Formulation is placed in a dialysis membrane, molecular weight cut-off=12-14 kDa, given in TABLE 4, Example 5).

FIG. 5 illustrates the in-vitro release profile of meloxicam from Formulations #6 and #7 into PBS, pH=7.4 (Formulation is placed in a dialysis membrane, molecular weight cut-off=12-14 kDa, given in TABLE 4, Example 5).

FIG. 6 illustrates the in-vitro release profile of sulfadiazine from Formulation #8 and #9 into PBS, pH=7.4 (Formulation is placed in a dialysis membrane, molecular weight cut-off=12-14 kDa, given in TABLE 5, Example 5).

FIG. 7 illustrates the in-vitro release profile of methotrexate from Formulation #10 and #11 into PBS, pH=7.4 (Formulation is placed in a dialysis membrane, molecular weight cut-off=12-14 kDa, given in TABLE 6 Example 5).

FIG. 8 illustrates the in-vitro release profile of meloxicam and bupivacaine from Formulation #1 into PBS, pH=7.4, with 10 wt. % of methanol (Formulation is placed in a dialysis membrane, molecular weight cut-off=12-14 kDa, given in TABLE 1, Example 2).

FIG. 9 illustrates the in-vitro release profile of meloxicam and bupivacaine from Formulation #14 into PBS, pH=7.4, with 10 wt. % of methanol (Formulation is placed in a dialysis membrane, molecular weight cut-off=12-14 kDa, given in TABLE 7, Example 6).

FIG. 10 illustrates the in-vitro release profile of levothyroxine from Formulation #3 into PBS, pH=7.4 (formulation is placed in a dialysis membrane, molecular weight cut-off=12 to 14 kDa, given in TABLE 3, Example 5).

FIG. 11 illustrates the in-vitro release profile of ropivacaine (13% drug loading (%)) from Formulation #28 into PBS, pH=7.4 (Formulation is placed in a hydroxypropyl methylcellulose (HPMC) capsule, given in TABLE 15, Example 9).

FIG. 12 illustrates the in-vitro release profile of meloxicam and bupivacaine from Formulation #2 into PBS, pH=7.4 (Formulation is placed in a hydroxypropyl methylcellulose (HPMC) capsule, given in TABLE 2, Example 4).

FIG. 13 illustrates the in-vitro release profile of meloxicam and bupivacaine from Formulation #2 into PBS, pH=7.4 (Formulation is placed in a dialysis membrane, molecular weight cut-off=12-14 kDa, given in TABLE 2, Example 4).

FIG. 14 illustrates the plot of time to reach 50% release (T₅₀ in hours) versus the water content (wt. %) of the formulation provided in TABLE 7, Example 6 (Formulation is placed in a dialysis membrane, molecular weight cut-off=12-14 kDa).

FIG. 15 illustrates the plot of time to reach 50% release (Tso in hours) versus egg phosphocholine (wt. %) of the formulation without water provided in TABLE 8, Example 6 (Formulation is placed in a dialysis membrane, molecular weight cut-off=12-14 kDa).

FIG. 16 illustrates the plot of time to reach 50% release (Tso in hours) versus egg phosphocholine (wt. %) of the formulation with 10% water provided in TABLE 9, Example 6 (Formulation is placed in a dialysis membrane, molecular weight cut-off=12-14 kDa).

FIG. 17 illustrates the plot of the in-vitro release profiles of different amounts of α-tocopherol (wt. %) of the formulation provided in TABLE 12, Example 8 (Formulation is placed in a dialysis membrane, molecular weight cut-off=12-14 kDa).

FIG. 18 illustrates the plot of the in-vitro release profiles of different low HLB oils of the formulation provided in TABLE 13 Example 8 (Formulation is placed in a dialysis membrane, molecular weight cut-off=12-14 kDa).

FIG. 19 illustrates the plot of the in-vitro release profiles of different amounts of RESOMER 505 (wt. %) of the formulation provided in TABLE 14, Example 8 (Formulation is placed in a dialysis membrane, molecular weight cut-off=12-14 kDa).

FIG. 20 illustrates the birefringence image from the polarized light microscope of Formulation #28 before the addition of water (left), and one day after the addition of water (right), as shown in TABLE 15, Example 9.

FIG. 21 illustrates the birefringence image from the polarized light microscope of Formulation #15. 5 minutes after the addition of water (left), and one day after the addition of water (right), as shown in TABLE 8, Example 6.

### DETAILED DESCRIPTION OF THE INVENTION

Before the present invention is further described, it is to be understood that this invention is not limited to the particular embodiments described. It is also to be understood that the terminology used herein is for describing particular embodiments only, and is not intended to be limiting.

It should be noted that, as used herein, the singular forms “a,” “an,” and “the” include plural referents unless the context clearly dictates otherwise.

Where a range of values is provided, it is understood that each intervening value, to the tenth of the unit of the lower limit, unless the context clearly dictates otherwise, between the upper and lower limit of that range and any other stated or intervening value in that stated range, is encompassed within the invention. The upper and lower limits of these smaller ranges may independently be included in the smaller ranges, and are encompassed within the invention, subject to any specifically excluded limit in the stated range. Where the stated range includes one or both of the limits, ranges excluding either or both of those included limits are also included in the invention.

The term ‘about’ generally refers to plus or minus 10% of the indicated number. For example, ‘about 10%’ may indicate a range of 9% to 11%, and “about 20” may mean from 18 to 22. Other meanings of ‘about’ may be apparent from the context, such as rounding off, so, for example, ‘about 1’ may also mean from 0.5 to 1.4. Similarly, ‘about 0.2’ may encompass the value 0.22.

The term “weight %” and “wt. %” are used interchangeably and, unless otherwise stated, are based on the total weight of the composition.

The terms “bioactive agent,” “pharmaceutical active ingredient,” and “drug” are used interchangeably unless otherwise stated.

As used herein the term “controlled release” and “sustained release” are used interchangeably and refers to a drug delivery system/dosage formulation that releases the desired amount of bioactive agent, pharmaceutical active ingredient (“API”) or drug molecule for a defined or extended period ranging from days to months, for example, in some embodiments, the release of about 80 to 90% of drug molecule in 5 to 15 days, and in some embodiments, release of about 30% of drug molecule in about 30 days, etc. The release of the drug molecule from the formulation of the invention can be in a continuous, discontinuous, linear, or nonlinear manner, preferably a continuous and linear manner.

As used herein, the term ‘preformulation’ or “precursor formulation” refers to a composition or formulation that can form a liquid crystal before or after injection. In certain embodiments, the preformulation or precursor formulation will comprise an ion pair comprising at least one bioactive agent, pharmaceutical active ingredient, or drug, one or more phospholipids, one or more low HLB lipids other than the phospholipid, and/or one or more bio-tolerated organic solvents. In certain aspects of this embodiment, the preformulation or precursor formulation will be free or substantially free of water. In certain aspects, the preformulation or precursor formulation may comprise one or more release adjusters or controlled release agents and/or one or more pharmaceutically acceptable excipients, such as surfactants/wetting agents, pH adjusting agents, tonicity agents, buffering agents, or combinations thereof.

As used herein, “substantially free” means less than 10%, 9%, 8%, 7%, 6%, 5%, 4%, 3% 2% or 1% of the stated ingredient. The term “free of” means the composition is free of detectable amounts of the stated ingredient.

As used herein, the term “liquid crystal formulation” or “liquid crystal composition” broadly encompasses the preformulation, precursor formulation, and the final formulation that may be administered to a subject or patient unless specifically stated or the context indicates otherwise.

As used herein, the terms ‘treating’ and ‘treatment’ refer to reversing, alleviating, inhibiting, or slowing the progress of the disease, disorder, or condition to which such terms apply, or one or more symptoms of such disease, disorder, or condition.

As used herein, the term ‘subject’ or ‘patient’ used herein refers to a human patient or a mammalian animal, such as cats, dogs, cows, horses, monkeys, or the like.

As used herein, the term “poorly soluble” includes the “sparing soluble,” “slightly soluble,” “very slightly soluble,” “practically insoluble,” and/or “insoluble” as these terms are defined by the United States Pharmacopeia (USP). For example, the USP defines “sparingly soluble” as requiring from 30 to 100 parts solvent to dissolve 1 part of solute and “very slightly soluble” as requiring from 1000 to 10,000 parts solvent to dissolve 1 part of solute.

Any terms in the present application, unless specifically defined, will take the ordinary meanings as understood by a person of ordinary skill in the art.

A liquid crystal composition with enhanced drug solubility in accordance with the present invention may be prepared and characterized to identify a formulation and process that will allow the ion pairs formed between bioactive agents and bioactive agents and/or bioactive agents and excipients to be incorporated into the liquid crystal composition to confer enhanced solubility and impart sustained release of one or more bioactive agents. The disclosed compositions are preferably a flowy liquid or solution for administration, preferably parenteral administration, and have a high capacity for drug solubilization by using a low to no content of oils, i.e., free or substantially free of oils. The ion pairs are preferably hydrophobic and produce a liquid crystal depot that exhibits a sustained in vitro release behavior. Furthermore, the liquid crystal preformulation or precursor formulation is physically and chemically stable at room temperature for a prolonged period of time. The enhanced chemical stability is believed to be attributed to the partitioning of the ion pairs formed inside the lipid mixture, thereby protecting the bioactive agent or drug from precipitation or degradation in the solution.

In one aspect, the ion pair interaction between the bioactive agent/drug and bioactive agent/drug or the bioactive agent/drug and excipients will increase the bioactive agent's/drug's solubility in the liquid crystal composition, preformulation, precursor formulation, or final injectable formulation.

In some embodiments, the ion pair interaction is a Lewis acid-base interaction, i.e., the interaction between electron donors and electron acceptors. It also includes hydrogen bonding, the interaction between counterions, the interaction between electron deficient-electron sufficient molecules, or the interaction between metal ions and ligands.

In some embodiments, the ion pair interaction can be the interaction between drugs or the interaction between drugs and excipients. The acid part of the ion pair can be a bioactive agent, an excipient, or a combination of both. The base part of the ion pair can be a bioactive agent, an excipient, or a combination of both. In certain aspects of the present invention, the ion pair interaction is preferably an interaction between acidic bioactive agents and basic bioactive agents/excipients.

In some embodiments, the non-limiting examples of the bioactive agents that can work as the acid part of the ion pair interaction are the poorly soluble NSAIDs, the acid form of a local anesthetic, sulfadiazine, methotrexate, levothyroxine, and the mixture of two or more thereof. The acidic bioactive agent can also be other bioactive ingredients that show the property of a Lewis acid.

NSAIDs are generally acidic compounds with variable hydrophobicity. In some embodiments, a poorly soluble NSAID is selected from the group of NSAIDs that are slightly soluble, very slightly soluble, practically insoluble, or insoluble in water as defined by the United States Pharmacopeia. This includes etodolac, diflunisal, mefenamic acid, cataflam, indomethacin, piroxicam, meloxicam, oxaprozin, fenoprofen, salsalate, celecoxib, nabumetone, sulindac, and ketoprofen, and the mixtures of two or more thereof.

In some embodiments, the acid forms of local anesthetics are selected from the group of bupivacaine hydrochloride, ropivacaine hydrochloride, lidocaine hydrochloride, procaine hydrochloride, chloroprocaine hydrochloride, tetracaine hydrochloride, cocaine hydrochloride, benzocaine hydrochloride, and the mixtures of two or more thereof.

In some embodiments, the non-limiting examples of the bioactive agent in the base part are local anesthetics such as the free base form of a local anesthetic selected from the group consisting of bupivacaine, ropivacaine, lidocaine, procaine, chloroprocaine, tetracaine, cocaine, benzocaine, and the mixtures of two or more thereof. The local anesthetics are generally considered weak bases with pKa higher than 7. The basic bioactive agent can also be other bioactive ingredients that show the property of Lewis base.

In some embodiments, the excipients as the base part should be organic bases with a pKa higher than their acidic drug counterparts. The preferred organic bases that can be used in the disclosed liquid crystal formulation are bio-tolerated amines with an aliphatic chain with at least 4 carbons. The non-limiting examples are one or a combination of dodecylamine, tridodecylamine, didodecylamine, n-octylamine, di-n-octylamine, tri-n-octylamine, tri-n-hexylamine, and di-n-hexylamine. If the bioactive ingredients are hydrophobic, the basic excipients can be other high pKa ions, not limited to one or a combination of pamoate salts, and deoxycholate salts.

In some preferred embodiments, the basic excipients are chosen from the bio-tolerant amine with the hydrophobic moiety. Long-chain bases have a higher affinity to the liquid crystal phase and therefore, make the drug release sustainedly. This is to better entrap the bioactive agents in the lyophobic phase.

In one aspect, the liquid crystal composition that can form a stable depot with water, herein solubility-enhanced liquid crystal, comprises: an ion pair with at least one poorly soluble drug, at least one phospholipid, preferably an unsaturated phosphocholine, at least one low HLB lipids other than the phospholipid, a bio-tolerant organic solvent to that can dissolve the lipids and the ion pair and optionally a release adjuster or controlled release agent.

In one embodiment, the ion pair is formed by acidic and basic bioactive ingredients. The said acidic bioactive ingredients comprise between 0 wt. % and 20 wt. % by weight of the composition and preferably about 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20% by weight of the injectable formulation, precursor formulation or preformulation or any range within the afore recited values, and said basic bioactive ingredients comprise between 0 wt. % to 20 wt. % by weight of the composition and preferably about 0.1%, to about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20% by weight of the injectable formulation, precursor formulation or preformulation or any range within the afore recited values with the proviso that the composition contains a therapeutic amount of at least one bioactive agent.

In one embodiment, the ion pair is formed by an acidic bioactive ingredient and a basic excipient. The said acidic bioactive ingredients comprise between 0.1 wt. % to 20 wt. % by weight of the composition and preferably about 0.1%, to about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20% by weight of the injectable formulation, precursor formulation or preformulation or any range within the afore recited values and the basic excipients comprise between 0 wt. % and 20 wt. % by weight of the composition and preferably about 0.1%, to about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20% by weight of the injectable formulation, precursor formulation or preformulation or any range within the afore recited values with the proviso that the formulation contains a therapeutic amount of at least one bioactive agent.

In some embodiments of the composition, the wt. % ratio of the acidic counter ion, such as NSAIDs, can reach up to 20%, and the basic counter ion, such as the free base of the local anesthetics, can reach up to 20%, which could meet the need for the small-volume long-term release. In the previous commercial product, the concentration of the NSAIDs was only 0.085% and the local anesthetic was 2.85%, which is dissolved in 7 ml of polymers and organic solvents. The increased drug dose will be enough for the long-term release from 3 days to one month with 0.5 to 3 ml of injection.

In some embodiments, an initial fast release of the drug, i.e., a cumulative release of up to about 30%, up to about 35%, up to about 40%, up to about 45%, up to about 50%, up to about 55%, or up to about 60% of the total drug in the composition, in the first day after administration of the composition to the subject or patient. The high initial release will be suitable for urgent pain management. On the contrary, commercially available products like EXPAREL® or ZYRELEF® have a delayed release on the first day, which will not be suitable for patients in need of immediate pain care.

In some embodiments, the release of the drug from the composition following administration is sustained for about 3, 4, 5, 6, 7, 8, 9, 10, 11, 12, 13, 14, 15, 16, 17, 18, 19, 20, 21, 22, 23, 24, 25, 26, 27, 28, 29, 30 or 31 days. In certain embodiments, the release is sustained for 3-7 days, about 2 weeks, or about one month.

In some embodiments, the phospholipid is a phosphocholine, preferably an unsaturated phosphocholine. Non-limiting examples of the phosphocholine include but are not limited to egg phosphocholine, soy phosphocholine, dioleoylphosphocholine, diolcoylphosphatidylcholine (DOPC), dimyristoylphosphatidylcholine (DMPC), dilauroylphosphatidylcholine (DLPC), or a mixture of the foregoing. The phospholipid should form a lamellar phase for sustained release of the bioactive agent or drug. This can also stabilize the bioactive agent or drug in the depot.

A Lewis acid-base pair can be dissolved in high concentrations in a properly selected organic solvent system. However, precipitation may form from the high-concentration solutions after some time, such as a few weeks. The interaction between the Lewis acid-base pair and a lipid phase comprising a phospholipid and optionally a low HLB lipid other than a phospholipid can stabilize the high concentration Lewis acid-base pair solution and prevent precipitation. The concentration of the phospholipid in the compositions may range from about 5 wt. % to about 99 wt. %, preferably about 7.5 wt. % to about 90 wt. %, more preferably about 10 wt. % to about 85 wt. % and most preferably about 12 wt. % to 80 wt. %. In certain embodiments, the wt. % of the phospholipid, preferably an unsaturated phosphocholine, will be at least 7.5 wt. % to about 50 wt. %, preferably about 10 wt. % to about 45 wt. % and more preferably about 12 wt. % to about 40 wt. % to fully stabilize the ion pairs in the lamellar phase. While phospholipid or other low HLB lipids provide good compatibility for the Lewis acid-base pair, the strong interaction between the liquid crystal phase and the Lewis acid-base pair will make the drug ion pairs hard to release.

In some embodiments, the compositions described herein will comprise one or more low HLB lipids other than the phospholipid. The compositions described herein may comprise from 0 wt. % to 80 wt. %, preferably about 2.5 wt. % to about 70 wt. %, more preferably about 5 wt. % to about 60 wt. % and most preferably about 7.5 wt. % to 50 wt. % of the composition or one or more low HLB lipids. In certain embodiments, the compositions comprise about 1 wt. % to 80 wt. %, preferably about 3 wt. % to about 60 wt. % and more preferably about 5 wt. % to about 50 wt. % of one or more low HLB lipids. The low HLB lipids used in the compositions described herein will have an HLB value below 12, 11, 10, 9, 8, 7, 6, 5, 4, 3, 2, 1, or any range within the afore-listed values. HLB measures the lipophilicity of the lipids, and a low HLB means the lipid is more lipophilic. A preferred low HLB lipid that is useful in the compositions described herein is glyceryl dioleate, and it will form a reverse micellar phase when contacted with water. Other low HLB lipids that may be used in the compositions described herein include, but are not limited to, oleic acid, castor oil, sesame oil, glyceryl monololeate, and a mixture of two or more. Some of the low HLB lipids, like oleic acid, can also form a Lewis acid-base interaction with the bioactive ingredients and better solubilize the drug product. The combined phases formed by the low HLB lipids and the phospholipid can compensate for the phase change caused by drug release.

In some embodiments, the compositions described herein comprise at least one organic solvent or a mixture of organic solvents. The organic solvent or mixture of organic solvents should be a solvent to fully dissolve the phospholipids and the low HLB lipids. Examples of the organic solvents include, but are not limited to, methanol, ethanol, isopropanol, n-butanol, t-butanol, benzyl alcohol, benzyl benzoate, N-methyl-2-pyrrolidone (NMP), polyethylene glycol (PEG) such as PEG400, propylene glycol (PG), dimethylsulfoxide (DMSO), dimethylacetamide (DMAC), and mixtures thereof. Neither of the counterparts of Lewis acid-base pairs needs to be fully dissolved in the solvent before mixing. Upon mixing, the bioactive counterions will form Lewis acid-base pairs immediately, which will drive more solid drugs to dissolve in the solution. In some embodiments, the organic solvent composition comprises about 0 wt. % to 99 wt. % preferably about 2.5 wt. % to about 70 wt. %, more preferably about 5 wt. % to about 60 wt. % and most preferably about 7.5 wt. % to 50 wt. % of the compositions. The percentage of the organic solvent depends on the solubility of the bioactive agent ion pairs.

In some embodiments, the compositions described herein may comprise a release adjuster or control release agent. The release adjuster or control release agent comprises 0 wt. % to 50 wt. %, preferably about 0.5 wt. % to about 25 wt. % and more preferably about 1 wt. % to about 20 wt. % of the composition. Examples of preferred release adjusters are α-tocopherol, poly(lactic-co-glycolic) acid (PLGA) polymer, and mixtures thereof. The PLGA polymers have a lactide/glycolide (L/G) ratio of 90:10 to 10:90, 40:60 to 60:40, and preferably 50:50 with either an ester end or an acid end. Specific examples of PLGA polymers that may be used in the injectable formulations include an L/G ratio of 90:10, 40:60, and 50:50. Suitable molecular weight of the PLGA polymers desirably range from about 4000 Dalton to 120,000 Dalton, preferably 6000 Dalton to 80,000 Dalton.

A variety of PLGA copolymers may be used in the compositions described herein, with the ratio depending in part on the rate of degradation desired. For example, a 50:50 PLGA polymer, containing 50% lactide and 50% glycolide, would be a relatively fast-degrading copolymer, while 75:25 PLGA degrades more slowly, and 85:15 and 90:10 even more slowly, due to the increased polylactide components. It is readily apparent that a suitable ratio of lactide:glycolide is easily determined by one of skill in the art. Based on these basic principles, varying lactide:glycolide ratios can be employed in the compositions described herein in order to achieve a desired release profile.

In certain embodiments, the molecular weights of the PLGA polymers used in the compositions described herein may range from 4 kDa and 120 kDa, preferably about 5 kDa to about 75 kDa, and the polymer chain end groups are carboxyl-terminated or ester-terminated.

In some embodiments, the compositions described herein may comprise a mixture of the PLGA polymers having different lactide to glycolide ratios, and/or different average molecular weights, and/or different chain-end groups. In one aspect of this embodiment, the compositions are prepared with a mixture of PLGA polymers that comprises at least one carboxyl-terminated PLGA polymer with a molecular weight of about 5 kDa to about 40 kDa. In a further aspect of this embodiment, the PLGA polymer is a mixture of at least one carboxyl-terminated PLGA polymer and at least ester-terminated PLGA polymer wherein the ratio of carboxyl-terminated PLGA polymer to ester-terminated PLGA polymer ranges from about 1:1 to about 10:1, preferably about 2:1 to about 9:1 and more preferably about 3:1 to about 8:1. In a still further aspect of this embodiment, the carboxyl-terminated PLGA polymer has a molecular weight of about 5 kDa to about 50 kDa, preferably about 6 kDa to about 45 kDa and more preferably about 7 kDa to about 40 kDa and the ester-terminated PLGA polymer has a molecular weight of about 20 kDa to about 100 kDa, preferably about 30 kDa to about 90 kDa and more preferably about 40 kDa to about 75 kDa. The following table describes a few commercially available PLGA polymers that may be used in the formulations of the present invention:

| | L/G | | | End | |
|---|---|---|---|---|---|
| PLGA | Ratio | Mw | | Group | Source |
| Resomer ® RG 502 | 50:50 | 7-17 | kDa | Ester | Sigma Aldrich |
| Resomer ® RG 502H | 50:50 | 7-17 | kDa | Carboxyl | Sigma Aldrich |
| Resomer ® RG 503H | 50:50 | 24-38 | kDa | Carboxyl | Sigma Aldrich |
| Resomer ® RG 505 | 50:50 | 54-69 | kDa | Ester | Evonik |
| Resomer ® RG 752H | 75:25 | 4-15 | kDa | Carboxyl | Sigma Aldrich |

Another aspect of the invention is related to a process of making the pharmaceutical injectable formulation, preformulation, or precursor formulation. The preparation process is not limited to the method discussed below. In some embodiments, a preferable one-pot process is employed to ensure each component gets fully dissolved. This comprises the steps of (i) dissolving at least one phospholipid in a solvent or solvent mixture, adding one or more low HLB lipids to the solution of step (i), and optionally a release adjuster and (iii) dissolving the Lewis acid-base pairs into the mixture of step (ii) and stirred/mixed until a uniform clear solution is formed.

The process may further comprise a step of sterilizing the liquid crystal composition, i.e., the preformulation or precursor formulation of step (iii). The sterilizing step may be accomplished by filtering the clear solution of step (iii) through a hydrophobic membrane having a pore size of about 0.2 μm or less, which is a great advantage over injectables with drug suspensions, which cannot be sterilized.

In some embodiments, the process may employ a solvent or cosolvent system to significantly lower the viscosity of the composition. Non-limiting examples of viscosity-lowering solvents or cosolvents are methanol, ethanol, isopropanol, acetonitrile, N-methyl pyrrolidone, propylene glycol, and dimethyl sulfoxide, and a mixture of two or more.

A further aspect of the present disclosure is directed to an in-vitro dissolution test method for the compositions disclosed herein. The in-vitro dissolution method comprises the steps of: (i) preparing a dissolution medium by mixing an organic solvent with a buffer aqueous solution and (ii) using a container with a defined shape to hold the liquid crystal compositions.

The in vitro dissolution method of the present invention is particularly useful for monitoring batch-to-batch variability as part of a quality control procedure; as a means for determining the bioequivalence of different formulations, and as a means for assessing the durability of a formulation (for example, the Storage Stability).

In one of the embodiments, the organic solvent composition of the dissolution media is chosen from the group of methanol, ethanol, cyclodextrin, PEG400, and a mixture of more than two thereof, which takes up 0 wt. % to 10 wt. % of the dissolution media.

Preferably, the buffer used in the in-vitro dissolution media is a phosphate-buffered saline (PBS) with a pH of 7.4. Other non-limiting examples of suitable buffers which can be included in the dissolution medium include citrate buffers, lithium lactate, sodium lactate, potassium lactate, calcium lactate, lithium phosphate, sodium phosphate, potassium phosphate, calcium phosphate, lithium maleate, sodium maleate, potassium maleate, calcium maleate, lithium tartarate, sodium tartarate, potassium tartarate, calcium tartarate, lithium succinate, sodium succinate, potassium succinate, calcium succinate, lithium acetate, sodium acetate, potassium acetate, calcium acetate, or mixtures thereof.

In some embodiments, the depot-holding container may be chosen from the group consisting of hydroxypropyl methylcellulose (HPMC) capsules, immersion cells, and dialysis bags. Suitable apparatus that may be used for carrying out the method of the present invention include, but are not limited to, known dissolution apparatus, for example, those described in USP 24, such as Apparatus 1 (basket test), Apparatus 2 (paddle test), and modifications thereof.

In some embodiments, the preferred method will be using a dialysis bag as a container for the compositions in the in-vitro release test. The molecular weight cut-off of the dialysis bag can be chosen from 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15 kDa, or any range within the aforementioned values.

In an aspect, some of the invented formulations will include water as a component and are mixed with the preformulation before administration.

In some embodiments, the liquid crystal compositions described herein may be provided in a sealed container, together with sterilized water in a separate or second sealed container. The administration of the final composition is performed after mixing the water with the liquid crystal composition, i.e., the precursor formulation or preformulation.

In some embodiments, sterilized water is mixed with the preformulation or precursor formulation just prior to administration, i.e. about 1, 2, 3, 4, 5, 6, 7, 8, 9, 10, 11, 12, 13, 14, 15, 16, 17, 18, 19, 20, 21, 22, 23, 24, 25, 26, 27 28, 29, or 30 minutes, or during administration to make the drug ion pairs and get fully released. The addition of water can make the preformulation or precursor formulation partially form a liquid crystal with lamellar phases. This is to prevent the aggregation of insoluble drugs when the depot formulation is injected into an environment with excess water. In some embodiments, the release curve will plateau early because of the aggregation of the drugs, and the addition of water can significantly reduce the aggregation and extend the release.

The viscosity of the final formulation may dramatically increase after adding water because of the formation of the liquid crystal. In some embodiments, this phenomenon is observed when the organic solvent is low in content. However, a proper lipid-to-solvent ratio is adjusted in the compositions described herein so that the viscosity of the liquid crystal after mixing with water is below 200 c.p. This makes the drug mixture able to be smoothly injected and readily circulate in the bloodstream without causing a vein blockade or any embolization.

In some embodiments, the viscosity of the described formulations ranges from 50 c. p. (Centipoise), 200 c. p., 500 c. p., 1000 c. p., 2000 c. p., 5000 c. p., and 8000 c. p. The viscosity of the described formulation will directly influence the filterability and injectability of the oily formulation. Preferably, the viscosity should be 50 c. p., 200 c. p., 500 c. p., and 1000 c. p.

In some embodiments, sterilized water is not required or included as a component. In these embodiments, the preformulation is the final formulation and is provided in a single sealed container and may be administered without the addition of water or other ingredients. In these embodiments, bioactive agents or drugs can also be released fully over time without water.

Another aspect of the invention relates to a process for treating or preventing disease in a patient, comprising administering a therapeutically effective amount of a liquid crystal composition as described herein to a patient in need thereof. The disclosed compositions are suitable for a variety of administration methods, like intravenous, intramuscular, topical, and preferably subcutaneous injection. For the subcutaneous injections, there is a maximum injection volume. To minimize the pain caused by the subcutaneous injection, the maximum volume generally accepted is around 1.5 ml, although volumes of up to 3 ml are well tolerated when injected in the abdomen. The volume limitation and the dose requirement put a threshold on the dissolvability of drugs in the subcutaneous formulation. The drug dose of the EXPAREL® is 133 mg of bupivacaine in a 10 ml injection for 3 days. If this dose is injected subcutaneously, the wt. % of the drug will be over 10 wt. %. However, by using the ion pair formulation mentioned in this invention, the wt. % can be easily achieved.

The compositions described herein can be used for the treatment and prevention of post-operative pain or any pain with inflammation, such as joint pain caused by arthritis. For post-operative pain management, the injection can be conducted immediately after the surgery or days after the surgery for urgent pain relief. For the treatment of the joint pain caused by arthritis, the administration comprises locally injecting the compositions into the subject's joint having arthritis. The arthritis can be rheumatoid arthritis, osteoarthritis, or juvenile idiopathic arthritis.

The compositions described herein can also be used for the treatment of psoriasis, systemic lupus erythematosus, mycosis fungoides, dermatomyositis, Pityriasis rubra pilaris, sarcoidosis, vasculitis, Crohn's disease, ulcerative colitis, multiple sclerosis, eczema, non-Hodgkin lymphoma (advanced stage), and non-metastatic osteosarcoma, and treatment of infections.

The compositions described herein can also be used for the treatment of thyroid diseases such as hypothyroidism, enlarged thyroid glands, and thyroid cancer.

The following examples are provided by way of illustration only and are by no means intended to be limiting.

### Example 1

### List of Drug Substances and Liquid Crystal Raw Materials Used in Formulation #1 to Formulation #21

### Drug Substances

1) Meloxicam: 4-hydroxy-2-methyl-N-(5-methyl-2-thiazolyl)-2H-1,2-benzothiazine-3-carboxamide-1,1-dioxide, Crystallized Powder, ACMEC biochemical.
2) Bupivacaine: 1-butyl-N-(2,6-dimethylphenyl)-2-piperidinecarboxamide, Crystallized Powder, Cayman Chemical.
3) Ropivacaine Hydrochloride: (2S)—N-(2,6-dimethylphenyl)-1-propylpiperidine-2-carboxamide, hydrochloride, crystallized Powder. Anhong Pharmaceutical.
4) Sulfadiazine: 4-amino-N-pyrimidin-2-ylbenzenesulfonamide, Sigma-Aldrich.
5) Methotrexate: (2S)-2-[[4-[(2,4-diaminopteridin-6-yl)methyl-methylamino]benzoyl]amino] pentanedioic acid, MedChemExpress.
6) Levothyroxine: (2S)-2-amino-3-[4-(4-hydroxy-3,5-diiodophenoxy)-3,5-diiodophenyl]propanoic acid, crystallized Powder, ACMEC biochemical

### Example 2

### Preparation of Acid-Base Pair Liquid Crystal Formulations

The pharmaceutical Formulation #1 of the present invention is prepared by the following procedure:

A lipid solution is prepared by dissolving 1.00 grams of the egg phosphocholine (EPC) (source: LIPOID E 80) in 1.00 g of ethanol (EtOH) (Absolute, source: EMPROVE® EXPERT, Ph. Eur., BP, ChP, JP, USP)

1.06 g of the glyceryl dioleate (GDO) (source: FUJIFILM Wako Chemicals USA), 2.06 g of the N-methyl pyrrolidone (source: TCI chemicals), and 0.68 g of the PEG 400 (source: TEX LAB SUPPLY NF/FCC EP-USP) were added to the solution and stirred to mix well.

0.30 g of meloxicam (acid) and 0.60 g of bupivacaine (base) were added to the solution and were dissolved after stirring for 60 minutes.

In the proposed formulation above, ethanol, N-methyl pyrrolidone, and PEG 400 are examples of solvents. Egg phosphocholine is an example of an unsaturated phospholipid. Glyceryl dioleate (GDO) is an example of a low HLB lipid. Meloxicam is an example of the acidic bioactive ingredient, and bupivacaine is an example of the basic bioactive ingredient. They will change accordingly based on different formulations.

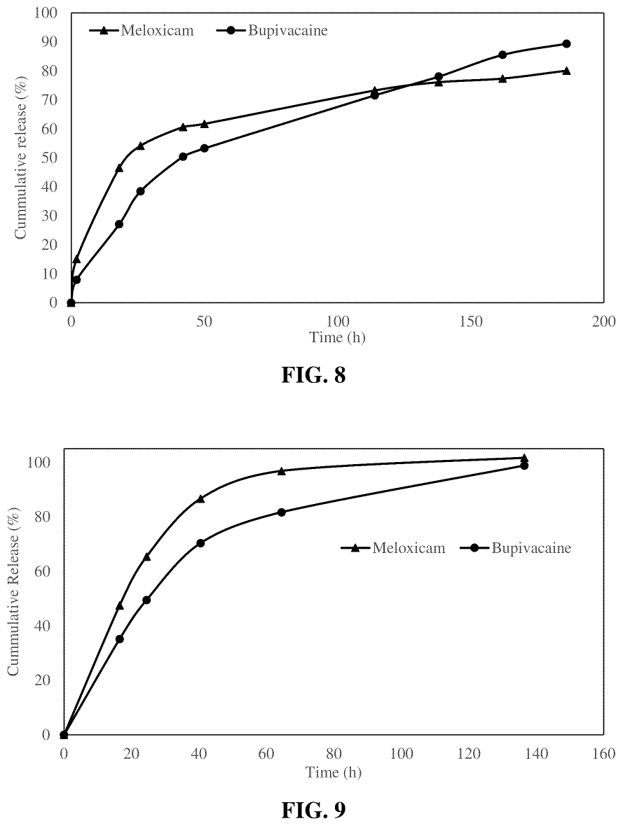

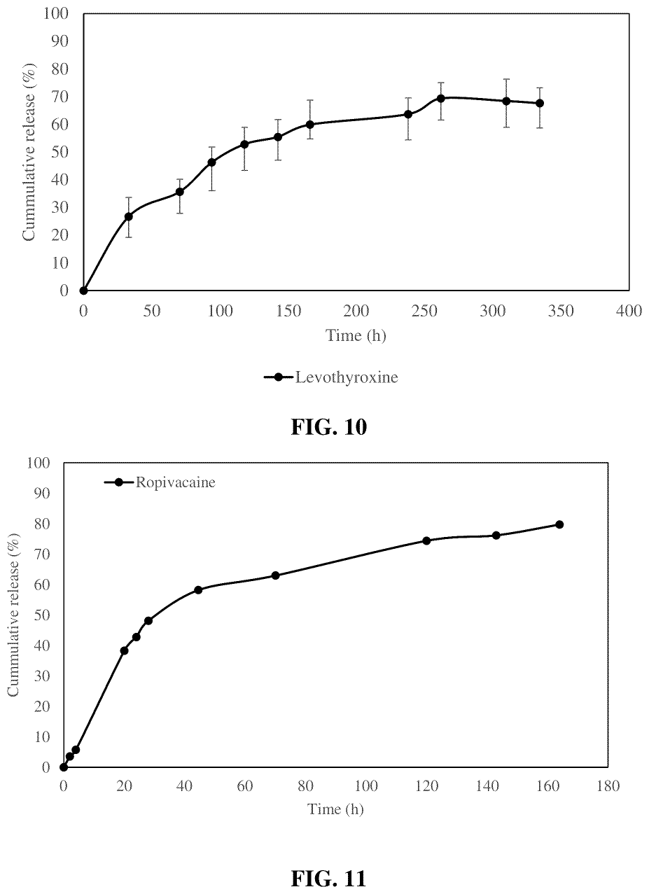

TABLE 1 provides some detailed information about Formulation #1, including the compositions of the raw materials. The cumulative release of the drugs is shown in FIG. 8.

TABLE 1

Formulation #1

| Ingredients | Name | Mass (g) | Weigt percentage |
|---|---|---|---|
| Unsaturated | EPC | 1.00 | 15% |
| phospholipid | | | |
| Low HLB | GDO | 1.06 | 17% |
| lipid | | | |
| Solvent | EtOH | 1.00 | 15% |
| | NMP | 2.06 | 31% |
| | PEG400 | 0.68 | 10% |
| Basic | Bupivacaine | 0.60 | 9% |
| drug/excipient | free base | | |
| Acidic | Meloxicam | 0.30 | 5% |
| drug/excipient | | | |

### Example 3

### High-Performance Liquid Chromatography (HPLC) Method for Determination of the Drug Loading Percentages in the Liquid Crystal Formulations

### Chromatographic Parameters:

Column: Phenomenex Luna C18 (2), 5 μm, 4.6 mm×250 mm
Column temperature: 25° C.
Mobile phase: acetonitrile/pH=7.6 phosphate buffer 60:40. (v/v)
Flow rate: 1.5 ml/min
Injection volume: 10 ul
Detector: UV at 220 nm for bupivacaine and UV at 360 nm for meloxicam.

### Chromatographic Parameters to Determine the Concentration of Levothyroxine:

Column: Phenomenex Luna C18 (2), 5 μm, 4.6 mm×250 mm
Column temperature: 25° C.
Mobile phase: acetonitrile/Water 35:65. (v/v), 0.1% phosphoric acid
Flow rate: 1.5 ml/min
Injection volume: 10 μl
Detector: UV at 302 nm

The standard solution was prepared by weighing approximately 100 mg of active pharmaceutical ingredients reference standard into a 100 ml volumetric flask, adding approximately 80 ml of ethanol, mixing and sonicating to dissolve, and making up to volume with ethanol. The stock standard solution was further diluted 1/10 with the mobile phase solution to make a working standard of approximately 100 μg/ml of each active pharmaceutical ingredient.

A sample solution was prepared by weighing approximately 100 mg of drug-loaded liquid crystal formulation into a 20 ml volumetric flask and adding ethanol to the volume. This solution was further diluted 1/20 with the mobile phase solution. The sample solution was filtered through a 0.45 μm polyvinylidene fluoride syringe filter before the HPLC injection.

The percent of drug loading is calculated by:

$$Drug Loading ( \% ) = Weight of bioactive ingredients in the Liquid Crystal Formulation / Total Weight of the Formulation .$$

### Example 4

### In Vitro Release Testing of Drug-Loaded Liquid Crystal Formulations and HPLC Method for Analyzing Drug Substances in In Vitro Release Samples

The in-vitro release testing of the drug-loaded liquid crystal was achieved by adding approximately 100 mg of the sample to a drug container, which can either be a molecular weight cut-off=12-14 kDa dialysis bag (Repligen Corp.) or a hydroxypropyl methylcellulose (HPMC) capsule (PURECAPS USA), and then putting it into 900 ml release medium. The release medium comprised the PBS buffer added with 10 wt. % of the methanol, and the pH was adjusted to about 7.40. The release medium was placed in a 1000 ml cylindrical glass jar (closed with a cap), which was placed in 37° C. water with a paddle spinning at a speed of 50 RPM. At each predetermined sampling time point, 1.5 ml of the medium was drawn from the dissolution media. The chromatographic parameters used are the same as in Example 3. The cumulative percentage of drug release is calculated based on the amount of the drug released into the total medium divided by the total amount of drug in the initial formulation.

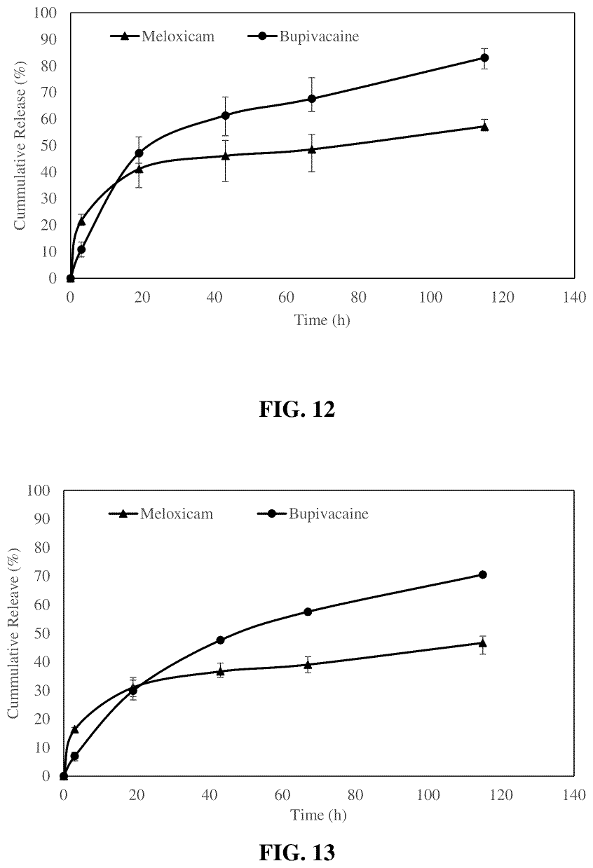

The comparison of the repetitiveness of the method by using a container of a molecular weight cut-off=12-14 kDa dialysis bag and an HPMC capsule in a sinker is shown in FIG. 11. & FIG. 12. The listed formulation in TABLE 2 is used for the method comparison. The preparation method is the same as Example 2, except for the amount of ingredients in the formulation. TABLE 2 shows the formulation for the comparison of the dissolution repetitiveness.

TABLE 2
Formulation #2

| Ingredients | Weight Percentage |
|---|---|
| EPC | 35 wt. % |
| GDO | 19 wt. % |
| EtOH | 9 wt. % |
| NMP | 15 wt. % |
| PEG400 | 6 wt. % |
| Bupivacaine | 9 wt. % |
| Free Base | |
| Meloxicam | 5 wt. % |

### Example 5

### Solubility Enhancing Effect and the Sustained Release Behavior of the Lewis Acid-Base Pair

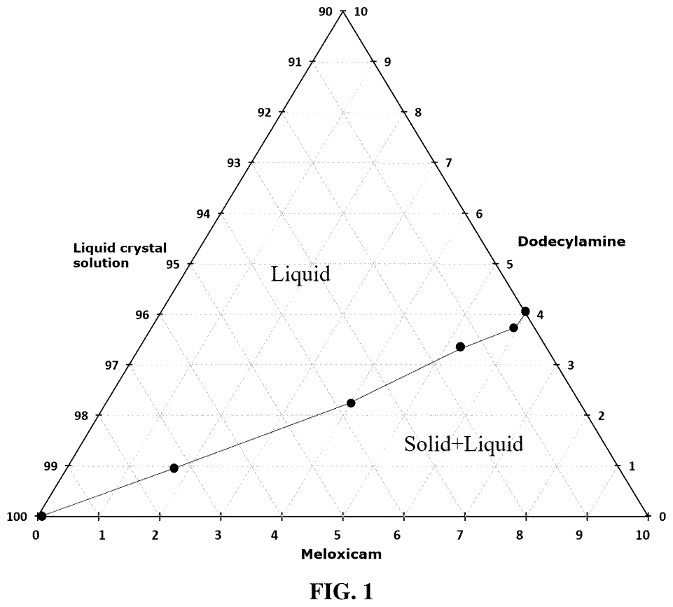

A ternary phase diagram plotted by meloxicam (wt. %), dodecylamine (wt. %), and the preformulated liquid crystal solution (composed of 30 wt. % of EPC, 50 wt. % of GDO, 7 wt. % of EtOH, 7 wt. % of NMP, and 5 wt. % of PEG400) is shown in FIG. 1.

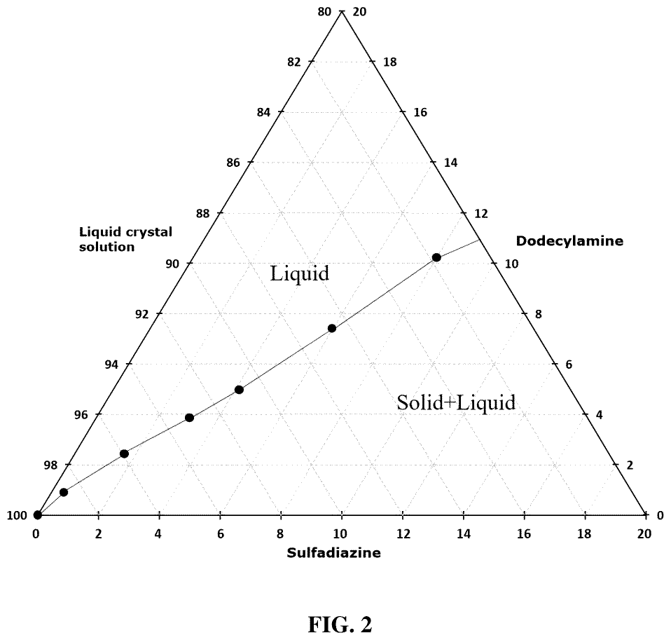

A ternary phase diagram plotted by sulfadiazine (wt. %), dodecylamine (wt. %), and the preformulated liquid crystal solution (composed of 30 wt. % of EPC, 50 wt. % of GDO, 7 wt. % of EtOH, 7 wt. % of NMP, and 5 wt. % of PEG400) is shown in FIG. 2.

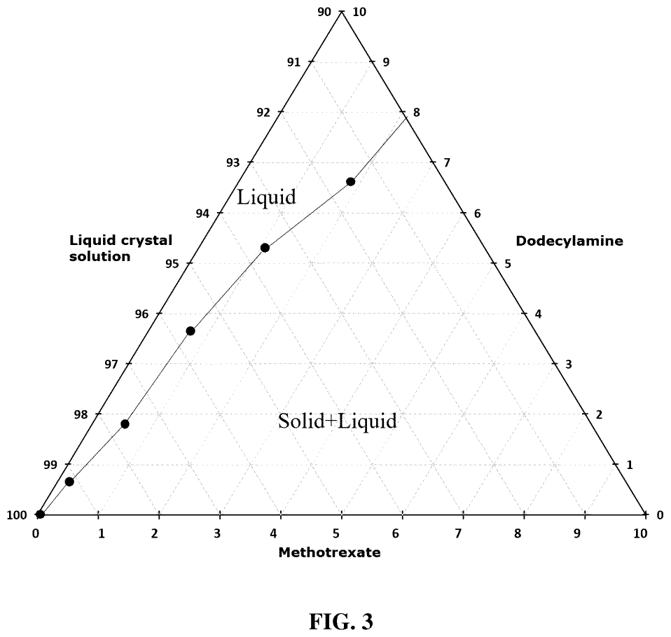

A ternary phase diagram plotted by methotrexate (wt. %), dodecylamine (wt. %), and the preformulated liquid crystal solution (composed of 30 wt. % of EPC, 50 wt. % of GDO, 7 wt. % of t-butanol, 7 wt. % of NMP, and 5 wt. % of PEG400) is shown in FIG. 3.

The solubility of the illustrated drugs in the solution without the counterions can only reach below 1%. However, the solubility will increase steadily with the addition of the dodecylamine.

The comparison between Formulation #3 and Formulation #4 as reported in TABLE 3 below, shows the solubility difference because of the addition of the dodecylamine. The drug loading percentage of the levothyroxine was determined by using the HPLC method mentioned in Example 3. With the addition of the dodecylamine, the opaque solution becomes clear, and the tested drug loading increases. This shows hydrophobic bases can enhance the solubility of the bioactive agents. Meanwhile, Formulation #3 shows a sustained release behavior.

TABLE 3

| Formulation | #3 | #4 |
|---|---|---|
| Ingredients | Mass | Mass |
| Unsaturated | EPC = 0.83 g | EPC = 0.83 g |
| Phospholipid | | |
| Low HLB Lipid | Glyceryl | Glyceryl |
| | linoleate = 1.1 g | linoleate = 1.1 g |
| Solvent | EtOH = 0.3 g | EtOH = 0.3 g |
| | NMP = 0.6 g | NMP = 0.6 g |
| | PEG400 = 0.2 g | PEG400 = 0.2 g |
| Basic excipient | Dodecylamine = 0.05 g | NA |
| Acidic Drug | Levothyroxine = 0.02 g | Levothyroxine = 0.02 g |
| Appearance | Clear | Opaque |
| Drug loading (%) | 0.51% | 0.41% |

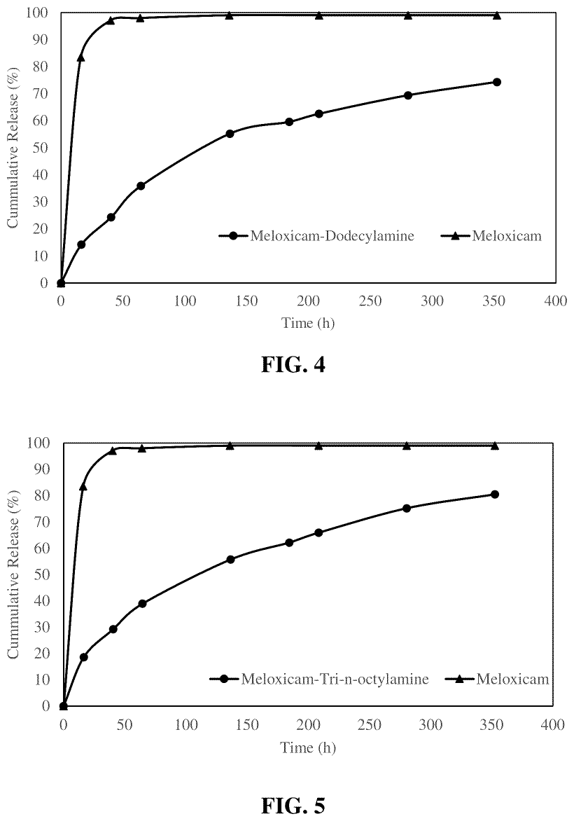

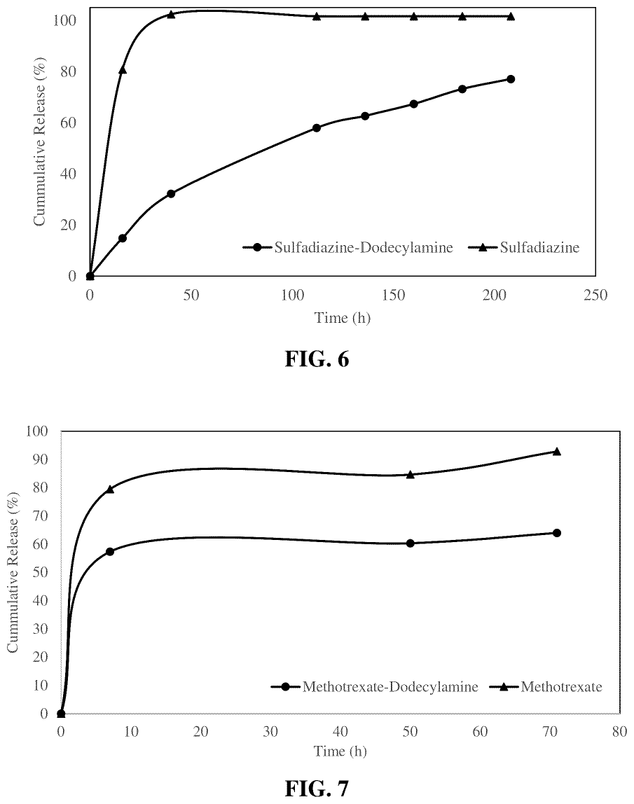

The formulations in TABLE 4 to TABLE 6 were prepared to show the enhanced sustained release behavior using the hydrophobic ion pair method. The drugs and excipients in Formulations #5, #6, and #7, Formulation #8 and #9, and Formulation #10 and #11 are the same except for the excipient counterions. An in-vitro release test was conducted for each formulation using the method in Example 4, and the results are shown in FIG. 4 to FIG. 7 The preformulation remained clear before the in-vitro release test. The formulations with the acidic/basic excipients will have a sustained release behavior. This is because the hydrophobic ion pair formed by the drugs and excipients will increase the compatibility of the drugs in the liquid crystal phase, and thus trap the drugs in the liquid crystal phase.

TABLE 4

| Formulation | #5 | #6 | #7 |
|---|---|---|---|
| Ingredients | Mass | Mass | Mass |
| Unsaturated | EPC = 0.50 g | EPC = 0.50 g | EPC = 0.50 g |
| Phospholipid | | | |
| Low HLB Lipid | GDO = 0.78 g | GDO = 0.78 g | GDO = 0.78 g |
| Solvent | EtOH = 0.25 g | EtOH = 0.25 g | EtOH = 0.25 g |
| | NMP = 1.17 g | NMP = 1.17 g | NMP = 1.17 g |
| Basic excipient | Dodecylamine = | Tri-n-Octylamine = | N/A |
| | 0.15 g | 0.20 g | |
| Acidic Drug | Meloxicam = 0.15 g | Meloxicam = 0.15 g | Meloxicam = 0.15 g |
| Appearance | Clear | Clear | Clear |

TABLE 5

| Formulation | #8 | #9 |
|---|---|---|
| Ingredients | Mass | Mass |
| Unsaturated | EPC = 0.50 g | EPC = 0.50 g |
| Phospholipid | | |
| Low HLB Lipid | GDO = 0.78 g | GDO = 0.78 g |
| Solvent | EtOH = 0.25 g | EtOH = 0.25 g |
| | NMP = 1.17 g | NMP = 1.17 g |
| Basic excipient | Dodecylamine = 0.15 g | N/A |
| Acidic Drug | Sulfadiazine = 0.1 g | Sulfadiazine = 0.1 g |
| Appearance | Clear | Clear |

TABLE 6

| Formulation | #10 | #11 |
|---|---|---|
| Ingredients | Mass | Mass |
| Unsaturated | EPC = 0.50 g | EPC = 0.50 g |
| Phospholipid | | |
| Low HLB Lipid | GDO = 0.78 g | GDO = 0.78 g |
| Solvent | t-butanol = 0.47 g | t-butanol = 0.47 g |
| | NMP = 1.1 g | NMP = 1.1 g |
| | PEG400 = 0.34 g | PEG400 = 0.34 g |
| Basic excipient | Dodecylamine = 0.05 g | N/A |
| Acidic Drug | Methotrexate = 0.05 g | Methotrexate = 0.05 g |
| Appearance | Clear | Clear |

### Example 6

### T₅₀ of the In-Vitro Release

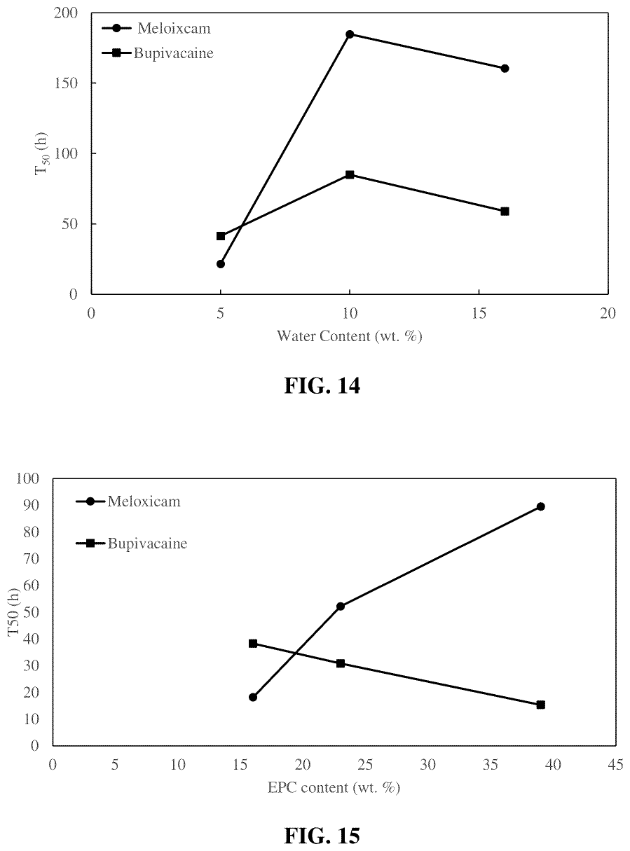

T₅₀ is the time to reach 50% release. While changing the formulations, the release curve will be different, and thus Tso changes. TABLE 7 to TABLE 9 provide some detailed information on Formulation #12 to #20, including the compositions of the raw materials and ion pair bioactive ingredients. The preparation method is the same as in Example 2, except for the amount of ingredients in the formulation. The T₅₀ of these formulations are shown in FIG. 14 to FIG. 16.

Formulation #12 to Formulation #14 are designed to investigate the release behavior because of the change in the formulation's water content. As the content of the water increases, the release time will be extended. The change in the meloxicam's release time is much more significant than that of the bupivacaine when changing the water content in the formulation. The appearance of the formulation will also become more blurred when more water is added. However, when the amount of water goes higher than 10 wt. %, the release rate will decrease. Due to the different water solubility for different drugs, the change will be different, and herein the change is more significant for the meloxicam than the bupivacaine.

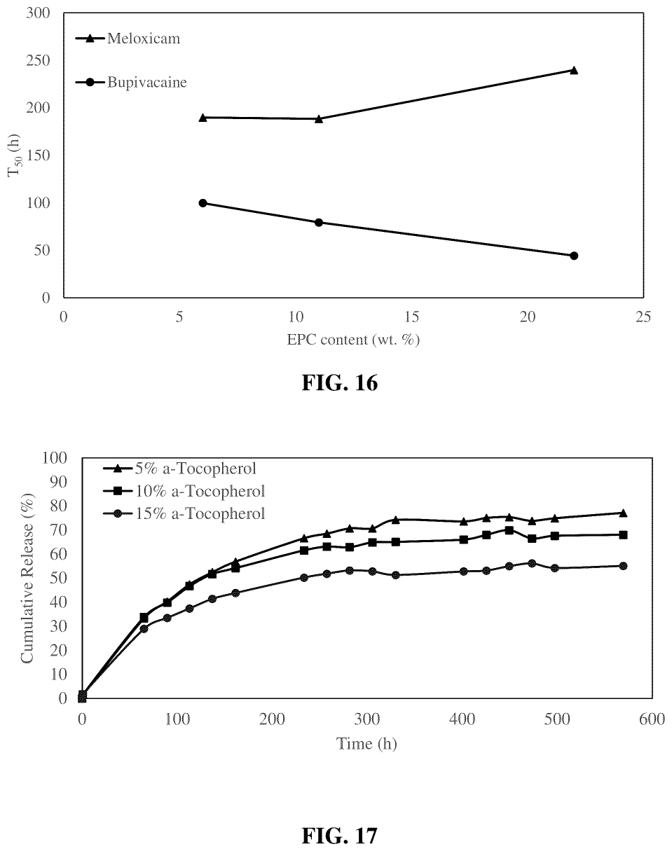

Formulation #15 to Formulation #20 are designed to investigate the release behavior because of the change in the EPC/GDO content in the formulation. TABLE 8 shows the formulations with the change in the EPC/GDO content (without water in the final injection), and TABLE 9 shows the formulations with the change in the EPC/GDO content (with water in the final injection). The Tso of the in-vitro release for the formulation with and without water is shown in FIG. 15 and FIG. 16. The responses of different drugs to the change in the EPC/GDO content are different. As the ratio of EPC/GDO gets higher, the release rate of the bupivacaine will get faster and faster. On the other hand, the release rate of the meloxicam will be thwarted. This discrepancy is mainly attributed to the differences in the lipophilicity of the drugs.

TABLE 7

| | Formulation | Formulation | Formulation |
|---|---|---|---|
| Ingredients | #12 | #13 | #14 |
| Water | 15 wt. % | 10 wt. % | 5 wt. % |
| EPC | 16 wt. % | 16 wt. % | 16 wt. % |
| Glyceryl | 7 wt. % | 12 wt. % | 19 wt. % |
| Dioleate | | | |
| EtOH | 12 wt. % | 12 wt. % | 12 wt. % |
| NMP | 23 wt. % | 23 wt. % | 23 wt. % |
| PEG400 | 11 wt. % | 11 wt. % | 11 wt. % |
| Bupivacaine | 9 wt. % | 9 wt. % | 9 wt. % |
| Meloxicam | 5 wt. % | 5 wt. % | 5 wt. % |

TABLE 8
Formulations with the Change of the EPC Content
(without water in the final injection)

| | Formulation | Formulation | Formulation |
|---|---|---|---|
| Ingredients | #15 | #16 | #17 |
| Water | 0 wt. % | 0 wt. % | 0 wt. % |
| EPC | 21 wt. % | 23 wt. % | 39 wt. % |
| Glyceryl | 19 wt. % | 16 wt. % | 0 wt. % |
| Dioleate | | | |
| EtOH | 12 wt. % | 12 wt. % | 12 wt. % |
| NMP | 23 wt. % | 23 wt. % | 23 wt. % |
| PEG400 | 11 wt. % | 11 wt. % | 11 wt. % |
| Bupivacaine | 9 wt. % | 9 wt. % | 9 wt. % |
| Meloxicam | 5 wt. % | 5 wt. % | 5 wt. % |

TABLE 9
Formulations with the Change of the EPC Content
(with water in the final injection)

| | Formulation | Formulation | Formulation |
|---|---|---|---|
| Ingredients | #18 | #19 | #20 |
| Water | 10 wt. % | 10 wt. % | 10 wt. % |
| EPC | 11 wt. % | 16 wt. % | 22 wt. % |
| Glyceryl | 18 wt. % | 13 wt. % | 7 wt. % |
| Dioleate | | | |
| EtOH | 12 wt. % | 12 wt. % | 12 wt. % |
| NMP | 23 wt. % | 23 wt. % | 23 wt. % |
| PEG400 | 11 wt. % | 11 wt. % | 11 wt. % |
| Bupivacaine | 9 wt. % | 9 wt. % | 9 wt. % |
| Meloxicam | 5 wt. % | 5 wt. % | 5 wt. % |

### Example 7

### Viscosity and Filterability of the Liquid Crystal Formulation

The viscosity of the liquid crystals was tested by Brookfield Viscometer DV-II+ Pro at a constant temperature (25° C.). The water was added at the start of the tests if water was included in the formulation. The measurements were conducted using a Helipath-type spindle at three rotational speeds: 3, 5, and 10 RPM.

TABLE 10. shows the viscosity of the formulation and the viscosity change after the addition of water (if water is included in the formulation). The viscosity of the formulations including the formulations can have a viscosity lower than 200 c.p., which is flowy enough for the 0.22 μm hydrophobic membrane filter sterilization. Drug Loading (%) before and after the injection through the polytetrafluoroethylene (PTFE) membranes (Titan3™) for the formulation is included in TABLE 11.

TABLE 10

| | Viscosity | Viscosity after adding water | | | |
|---|---|---|---|---|---|
| | before | 0 | 15 | 60 | 180 |
| | adding water | minutes | minutes | minutes | minutes |
| Formulation | 10.4 c.p. | 9.17 c.p. | 71.2 c.p. | 11.3 c.p. | 16.2 c.p. |
| #12 | | | | | |
| Formulation | 20.7 c.p. | 14.6 c.p. | 37.2 c.p. | 43.7 c.p. | 45.3 c.p. |
| #13 | | | | | |
| Formulation | 23.9 c.p. | 24.3 c.p. | 34.0 c.p. | 22.6 c.p. | 38.8 c.p. |
| #14 | | | | | |
| Formulation | 17.0 c.p. | 29.1 c.p. | 21.8 c.p. | 34.0 c.p. | 34.0 c.p. |
| #18 | | | | | |
| Formulation | 21.8 c.p. | 24.3 c.p. | 34.0 c.p. | 22.6 c.p. | 38.8 c.p. |
| #19 | | | | | |
| Formulation | 29.1 c.p. | 72.8 c.p. | 24.3 c.p. | 38.8 c.p. | 133.5 c.p. |
| #20 | | | | | |

TABLE 11

| | Bupivacaine drug loading | | Meloxicam drug loading | |
|---|---|---|---|---|
| | percentage (%) | | percentage (%) | |
| | Before | After | Before | After |
| | filtration | filtration | filtration | filtration |
| Formulation | 8.7 | 8.6 | 4.6 | 4.5 |
| #15 | | | | |
| Formulation | 8.7 | 8.8 | 4.3 | 4.4 |
| #16 | | | | |
| Formulation | 8.6 | 8.5 | 4.4 | 4.3 |
| #17 | | | | |

### Example 8

### Function of α-Tocopherol and Different Low HLB Lipids

Formulation #21 to #23 were prepared using the method shown in Example 2 to compare the effect of the amount of α-tocopherol on the in-vitro release profile of the liquid crystal formulation. The in vitro release of the formulations with α-tocopherol is shown in FIG. 17. The addition of α-tocopherol can slow down the release rate of the meloxicam. This could be attributed to the high hydrophobicity and high viscosity of the α-tocopherol.

TABLE 12
Formulations with the Change of the α-Tocopherol Content

| | Formulation | Formulation | Formulation |
|---|---|---|---|
| Ingredients | #21 | #22 | #23 |
| α-tocopherol | 5 wt. % | 10 wt. % | 15 wt. % |
| EPC | 48 wt. % | 46 wt. % | 43 wt. % |
| Maisine CC | 35 wt. % | 33 wt. % | 31 wt. % |
| EtOH | 8 wt. % | 8 wt. % | 8 wt. % |
| Dodecylamine | 1 wt. % | 1 wt. % | 1 wt. % |
| Meloxicam | 1 wt. % | 1 wt. % | 1 wt. % |

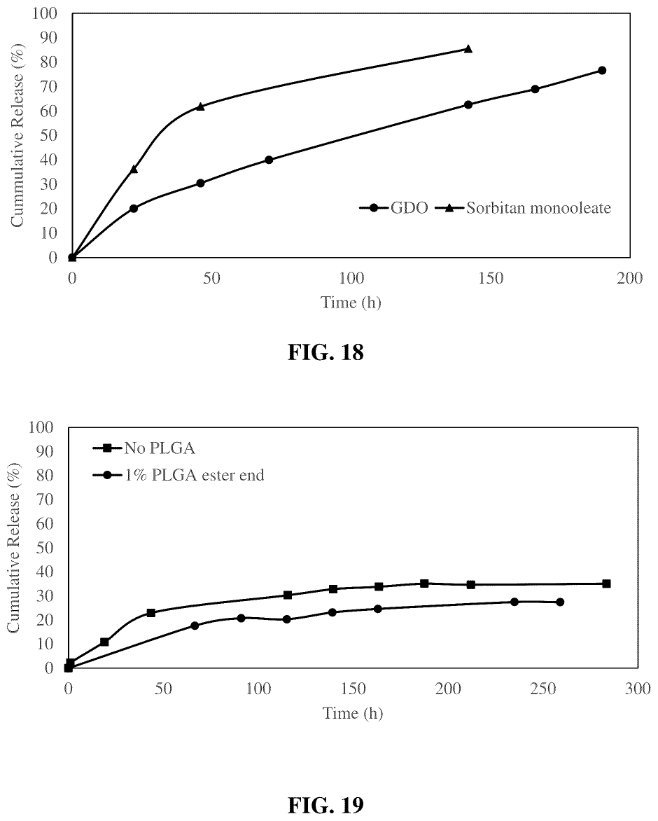

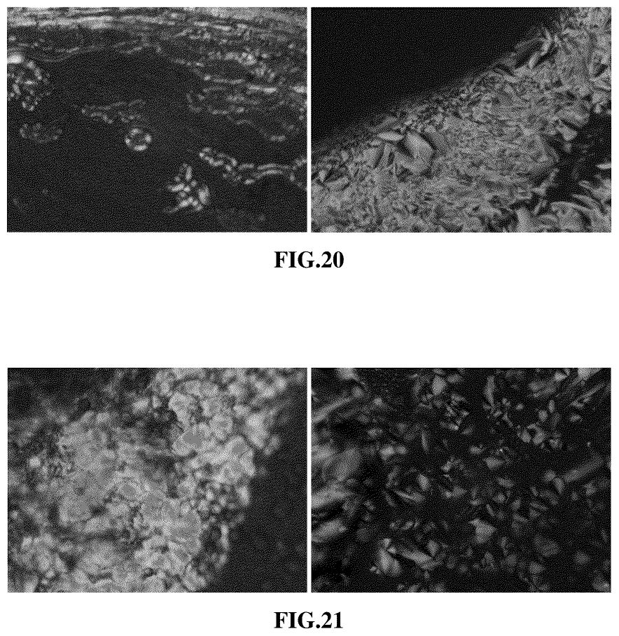

Formulation #24 and #25 were prepared to compare the effect of different HLB lipids on the in vitro release. Sorbitan monooleate and glyceryl dioleate (GDO) were added to the liquid crystal formulation to compare the effect of different low HLB lipids. These lipids have different HLB values and different viscosities; therefore, they show different in vitro release behavior. Although sorbitan monooleate has a higher viscosity, however, the release rate is high because of the high HLB. The in vitro release of formulations Formulation #24 and #25 are shown in FIG. 18.

TABLE 13
Formulations With the Change of Different Low HLB Oils

| | Formulation | Formulation |
|---|---|---|
| Ingredients | #24 | #25 |
| α-tocopherol | 15 wt. % | 15 wt. % |
| EPC | 66 wt. % | 66 wt. % |
| Low HLB | GDO | Sorbitan monooleate |
| lipids | 8 wt. % | 55 wt. % |
| EtOH | 8 wt. % | 8 wt. % |
| Dodecylamine | 1 wt. % | 1 wt. % |
| Meloxicam | 1 wt. % | 1 wt. % |

Formulation #26 and #27 were prepared to compare the effect of the addition of PLGA on the in-vitro release profile. As reported in doi: 10.1016/j.ijpharm.2024.125080, the ester end PLGA will result in longer release time of the meloxicam. The result reported herein shows the same trend as when an ester-end PLGA was added to the liquid crystal implant. This may be attributed to the increased viscosity because of the addition of a hydrophobic polymer composition. The in vitro release of formulations Formulation #26 and #27 are shown in FIG. 19.

TABLE 14
Formulations With the Change of the PLGA Content

| | Formulation | Formulation |
|---|---|---|
| Ingredients | #26 | #27 |
| α-tocopherol | 42 wt. % | 42 wt. % |
| EPC | 43 wt. % | 43 wt. % |
| Mainsine CC | 5 wt. % | 5 wt. % |
| EtOH | 5 wt. % | 5 wt. % |
| Dodecylamine | 1 wt. % | 1 wt. % |
| Meloxicam | 1 wt. % | 1 wt. % |
| Resomer 505 | 1 wt. % | 0 wt. % |

### Example 9

### Polarized Light Microscope

Phase behavior of the liquid crystal mesophases before and after the addition of water was characterized by polarized light microscopy. One drop of the formulation prepared by TABLE 15. was added onto a microscope slide. Water was added at the start of the observation. The liquid crystal textures were observed under a polarizing microscope by Radical Scientific Equipments Pvt. Ltd. that was fitted with a camera at ambient temperature. A magnification of ×200 was used. The crystallized structure will show a birefringence under polarized light, which can be observed by the microscope. The self-assembly of the liquid crystals from the oil droplets can be observed under the microscope.

TABLE 15
Formulation #28

| Ingredient | Weight % |
|---|---|
| Water | 30 wt. % |
| Ropivacaine | 13 wt. % |
| hydrochloride | |
| Sodium | 17 wt. % |
| Lauryl sulfate | |
| EPC | 16 wt. % |
| GDO | 14 wt. % |
| Ethanol | 11 wt. % |

### Example 10

### Stability of the Liquid Crystal Formulations without Water

The formulations without water on TABLE 16 were stored at room temperature until the inspection was completed. Formulation #29 shows precipitation with a low concentration of EPC after two weeks. However, when the EPC amount goes high, the precipitation disappears. The content of EPC in the formulation will control the stability of the formulations. When the EPC is below 12 wt. % in the Formulation, there will be precipitation coming out from the solution over months. After centrifuging, the precipitation is analyzed by using the HPLC method in Example 3. The molar equivalence of the bupivacaine and meloxicam in the precipitation is around 1:1.

TABLE 16

| | Formulation | Formulation |
|---|---|---|
| Ingredients | #29 | #30 |
| Water | 0 wt. % | 0 wt. % |
| EPC | 11 wt. % | 23 wt. % |
| Glyceryl | 29 wt. % | 19 wt. % |
| Dioleate | | |
| EtOH | 12 wt. % | 12 wt. % |
| NMP | 23 wt. % | 23 wt. % |
| PEG400 | 11 wt. % | 11 wt. % |
| Bupivacaine | 9 wt. % | 9 wt. % |
| Meloxicam | 5 wt. % | 5 wt. % |
| Precipitation | Yes | No |

The invention described herein may be practiced in the absence of any element or limitation which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A composition comprising:
a) a liquid crystal comprising a drug-drug ion pair or drug-excipient ion pair wherein the ion pair is a Lewis acid-base interaction;
b) about 5 wt. % to about 99 wt. % of one or more phospholipids;
c) 0 wt. % to about 80 wt. % percent of one or more low hydrophilic-lipophilic balance (HLB) lipids wherein the low HLB lipids exhibit an HLB value of 12 or less and are not a phospholipid;
d) 0 wt. % to about 99 wt. % of one or more bio-tolerant organic solvents; and
e) 0 wt. % to about 50 wt. % of a release adjuster
wherein a drug in the liquid crystal comprises about 0.1 wt. % to about 20 wt. % of the composition.

2. The ion pair of claim 1, wherein the interaction is between at least one acidic drug and at least one basic drug or at least one acidic drug and at least one basic excipient.

3. The ion pair of claim 2, wherein the at least one acidic drug is selected from the group consisting of a local anesthetic, a poorly soluble NSAID, sulfadiazine, methotrexate, levothyroxine, and mixtures thereof.

4. The ion pair of claim 2, wherein the at least one acidic drug is selected from the group consisting of etodolac, diflunisal, mefenamic acid, cataflam, indomethacin, piroxicam, meloxicam, oxaprozin, fenoprofen, salsalate, cetecoxib, nabumetone, sulindac, and ketoprofen, and mixtures thereof.

5. The ion pair of claim 2, wherein the at least one acidic drug is selected from the group consisting of bupivacaine hydrochloride, ropivacaine hydrochloride, lidocaine hydrochloride, procaine hydrochloride, chloroprocaine hydrochloride, tetracaine hydrochloride, cocaine hydrochloride, benzocaine hydrochloride, and mixtures thereof.

6. The ion pair of claim 2, wherein the at least one basic drug is selected from the group consisting a local anesthetic.

7. The ion pair of claim 6, wherein the local anesthetic is selected from the group consisting of bupivacaine, ropivacaine, lidocaine, procaine, chloroprocaine, tetracaine, cocaine, benzocaine, and mixtures of thereof.

8. The ion pair of claim 2, wherein the at least one basic excipient is an organic base.

9. The ion pair of claim 8, wherein the organic base is an amine with at least 4 carbons.

10. The ion pair of claim 9 wherein the amine is selected from the group consisting of dodecylamine, tridodecylamine, didodecylamine, n-octylamine, di-n-octylamine, tri-n-octylamine, n-hexylamine, tri-n-hexylamine, and di-n-hexylamine and mixtures thereof.

11. The ion pair of claim 8, wherein at least one basic excipient is a pamoate salt, a deoxycholate salt, and mixtures thereof.

12. The composition of claim 1 comprising:
about 7.5 wt. % to about 90 wt. % of one or more phospholipids;
about 2.5 wt. % to about 70 wt. % percent of one or more low HLB lipids;
about 2.5 wt. % to about 70 wt. % of one or more bio-tolerant organic solvents; and
0 wt. % to about 50 wt. % of a release adjuster.

13. The composition of claim 1 comprising:
about 10 wt. % to about 85 wt. % of one or more phospholipids;
about 5 wt. % to about 60 wt. % percent of one or more low HLB lipids;
about 5 wt. % to about 60 wt. % of one or more bio-tolerant organic solvents; and
0 wt. % to about 50 wt. % of a release adjuster.

14. The composition of claim 1 comprising:
about 12 wt. % to about 80 wt. % of one or more phospholipids;
about 7.5 wt. % to about 50 wt. % percent of one or more low HLB lipids;
about 7.5 wt. % to about 50 wt. % of one or more bio-tolerant organic solvents; and
0 wt. % to about 50 wt. % of a release adjuster.

15. The composition of claim 1 wherein the phospholipid is an unsaturated phosphocholine selected from the group consisting of egg phosphocholine, soy phosphocholine, dioleoylphosohcholine, and mixtures thereof.

16. The composition of claim 1 wherein the one or more bio-tolerant solvents are selected from the group consisting of methanol, ethanol, isopropanol, n-butanol, t-butanol, benzyl alcohol, benzyl benzoate, N-methyl-2-pyrrolidone (NMP), polyethylene glycol 400 (PEG400), propylene glycol (PG), dimethylsulfoxide (DMSO), dimethylacetamide (DMAC), water, and mixtures thereof.

17. The composition of claim 11 wherein the low HLB lipids are selected from the group consisting of glyceryl dioleate, glyceryl monololeate, oleic acid, castor oil, sesame oil, sunflower oil, and mixtures thereof.

18. A composition comprising:
a) a liquid crystal comprising a drug-drug ion pair or drug-excipient ion pair wherein the ion pair is a Lewis acid-base interaction and the ion pair is selected from: (i) at least one acidic drug selected from the group consisting of etodolac, diflunisal, mefenamic acid, cataflam, indomethacin, piroxicam, meloxicam, oxaprozin, fenoprofen, salsalate, cetecoxib, nabumetone, sulindac, and ketoprofen, sulfadiazine, methotrexate, levothyroxine, and mixtures thereof and a basic drug selected from the group consisting of bupivacaine, ropivacaine, lidocaine, procaine, chloroprocaine, tetracaine, cocaine, benzocaine, and mixtures thereof or (ii) at least one acidic drug selected from the group consisting of etodolac, diflunisal, mefenamic acid, cataflam, indomethacin, piroxicam, meloxicam, oxaprozin, fenoprofen, salsalate, cetecoxib, nabumetone, sulindac, and ketoprofen, sulfadiazine, methotrexate, levothyroxine, and mixtures thereof and a basic amine excipient selected from the group consisting of amine is selected from the group consisting of dodecylamine, tridodecylamine, didodecylamine, n-octylamine, di-n-octylamine, tri-n-octylamine, n-hexylamine, tri-n-hexylamine, di-n-hexylamine and mixtures thereof;
b) about 10 wt. % to about 85 wt. % of one or more unsaturated phosphocholines selected from the group consisting of egg phosphocholine, soy phosphocholine, dioleoylphosohcholine, and mixtures thereof;
c) about 5 wt. % to about 60 wt. % percent of one or more low hydrophilic-lipophilic balance (HLB) lipids wherein the low HLB lipids exhibit an HLB value of 12 or less, are not a phospholipid and are selected from the group consisting of glyceryl dioleate, glyceryl monololeate, oleic acid, castor oil, sesame oil, sunflower oil, and mixtures thereof;
d) about 5 wt. % to about 60 wt. % of one or more bio-tolerant organic solvents selected from the group consisting of methanol, ethanol, isopropanol, n-butanol, t-butanol, benzyl alcohol, benzyl benzoate, N-methyl-2-pyrrolidone (NMP), polyethylene glycol 400 (PEG400), propylene glycol (PG), dimethylsulfoxide (DMSO), dimethylacetamide (DMAC), water, and mixtures thereof; and
e) 0 wt. % to about 50 wt. % of a release adjuster
wherein a drug in the liquid crystal comprises about 0.1 wt. % to about 20 wt. % of the composition.

19. A method for preparing the composition of claim 1 comprising the steps of: dissolving one or more phospholipids in at least one bio-tolerant solvent to form a clear solution; combining the clear solution with one or more of the low HLB lipids and the ion pair.

20. An in-vitro dissolution test method for testing the composition of claim 1 comprising the steps of: preparing a dissolution medium by mixing an organic solvent with a buffered aqueous solution; filing a container with a defined shape with the composition to hold the composition during the duration of the in-vitro test and immersing the filled container in the dissolution medium for testing.

21. The method of claim 20 wherein the buffer for the aqueous solution is selected from the group consisting of citrate buffers, lithium lactate, sodium lactate, potassium lactate, calcium lactate, lithium phosphate, sodium phosphate, potassium phosphate, calcium phosphate, lithium maleate, sodium maleate, potassium maleate, calcium maleate, lithium tartarate, sodium tartarate, potassium tartarate, calcium tartarate, lithium succinate, sodium succinate, potassium succinate, calcium succinate, lithium acetate, sodium acetate, potassium acetate, calcium acetate, and mixtures thereof.

22. The method of claim 20, wherein the container is selected from the group consisting of hydroxypropyl methylcellulose (HPMC) capsules, immersion cells, and dialysis bags.

23. A method for treating a subject in need of treatment comprising administering the composition of claim 1 to the subject by intravenous injection, intramuscular injection, subcutaneous injection, or topical ointment.

24. The method of claim 23, wherein the subject is being treated for post-operative pain, local inflammation, joint pain, psoriasis, systemic lupus erythematosus, mycosis fungoides, dermatomyositis, Pityriasis rubra pilaris, or eczema.

25. The method of claim 24, wherein a single administration of the composition of claim 1 provides a therapeutic effective for 3 to 30 days.
